(19) United States
(12) Patent Application Publication
(10) Pub. No.: US 2010/0235931 A1
(43) Pub. Date: Sep. 16, 2010

(54) Novel Neurological Function of mPKCI

(76) Inventor: Jia Bei Wang, Towson, MD (US)

Correspondence Address: University of Maryland, Baltimore/Pratt, 620 W. Lexington Street, 4th floor, Baltimore, MD, 21201, US

(21) Appl. No.: 12/223,150

(22) PCT Filed: Feb. 9, 2007
(86) PCT No.: PCT/US07/03497

§ 371 Date: Jul. 23, 2008

Related U.S. Application Data
(60) Provisional application No. 60/771,779, filed on Feb. 9, 2006.

(51) Int. Cl. A01K 67/033; C12Q 1/68; A61K 31/7088; A61K 31/711; A61K 38/02; A61K 39/395; A61P 25/18

(52) U.S. Cl. 800/9; 435/6; 514/44 R; 514/44 A; 514/2; 424/130.1

(57) ABSTRACT

Wildtype and mice lacking the gene encoding PKCI/HINT 1 (PKC^{-/-}) were used to assess the involvement of PKCI/HINT1 in regulating basal locomotor activity and the behavioral activating effects of the psychostimulant, amphetamine. PKCl^{-/-} mice displayed low level of spontaneous locomotion relative to WT littermates. Acute administration of amphetamine significantly increased locomotor activity in WT mice; an effect that was enhanced in PKCl^{-/-} mice. Microdialysis studies revealed no alteration in basal DA dynamics in the striatum and nucleus accumbens of KO mice. Similarly, the ability of acute amphetamine to increase DA levels in these brain regions was unaltered. However, a dopamine receptor agonist, apomorphine (10 mg/kg), was able to induce a significantly higher locomotor activity in PKCI^{-/-} mice as compared with WT, suggesting there may be a dopaminergic functional change at the postsynaptic site. Our results also revealed that PKCI KO mice showed a less depression and anxiety trait than their litter mate controls (WT), which indicate that PKCI could also play a role in regulating the emotion states of brain. Together, these results indicated that PKCI/HINT1 may have a suppressive role in normal DA neurotransmission, and may also play an important role for the action of psychostimulants in schizophrenia.

This invention was made with government support under NIH Grant nos. DA11925 and DA018722. The government has certain rights in the invention.

### INTRODUCTION

PKCI/HINT1 is a ubiquitous member of the histidine triad (HIT) protein family that is characterized by the presence of a conserved HIT (HisXHisXHis, X is a hydrophobic amino acid) sequence motif (Klein et al., 1998, Exp. Cell Res 244, 26-32). The amino acid sequences of the members of this family are well conserved in a broad range of organisms including mycoplasm, plants, and mammals. PKCI/HINT1 protein is also widely expressed in rodent brain tissue including mesolimbic and mesostriatal regions. The murine PKCI/HINT1 (mPKCI/HINT1) is expressed at relatively high levels in several murine tissues, such as brain, liver and kidney. Little is known about the physiological role of PKCI/HINT1 proteins. Bovine PKCI (bPKCI) was originally identified as an in vitro inhibitor of PKC isoforms (McDonald and Walsh, 1985, Biochem. Biophys. Res. Commun. 129, 603-10). However, subsequent studies have questioned the physiological relevance of bPKCI as an in vivo inhibitor of PKC, and the interaction between PKCI and PKC (Klein et al., 1998, supra). Studies using X-ray crystallography and in vitro enzyme assays have elucidated the structural and functional aspects of PKCI/HINT1, suggesting a possible nucleotidyl hydrolase or transferase activity (Lima et al., 1997, Science 278, 286-90). PKCI/HINT1 has also been shown to interact with the ataxia-telangiectasia group D (ATDC) protein and the mi transcription factor in the yeast two-hybrid system (Brzoska et al., 1995, Genomics 36, 151-56). Recent studies indicate that PKCI/Hint1 knockout mice display increased susceptibility to carcinogenicity, suggesting that PKCI/HINT1 may normally play a tumor suppressor role (Su et al., 2003, Proc. Natl. Acad. Sci. USA 100, 7824-29). PKCI/HINT1 was also identified to specifically interact with the C-terminus of the mu opioid receptor (MOR) via a yeast two hybrid screening (Guang et al, 2004, Mol. Pharmacol. 66, 1285-92). This interaction led to an attenuation of receptor desensitization and inhibition of PKC-induced MOR phosphorylation. Furthermore, a deficiency in the expression of mPKCI in mice significantly enhanced both basal and morphine induced analgesia and caused a greater extent of tolerance to morphine. However, the definitive function of PKCI/HINT1 remains unknown.

Recently, PKCI/HINT1 was identified as one of the candidate molecules in the neuropathology of schizophrenia via microarray analysis (Vawter et al, 2001, Brain Res Bull. 2001 Jul. 15; 55(5):641-50. Vawter et al 2002, Schizophrenia Research 58 (2002) 11-20). This gene showed a fairly robust decrease in expression in the cortical samples from patients with schizophrenia compared with the match controls. And this change had also been validated by real-time quantitative polymerase chain reaction (Vawter et al 2004, Neurochem. Res. 29, 1245-55). Schizophrenia is a complex human disorder with unknown etiology. Several hypotheses have been proposed to explain the neurobiology of this disease (Lewis and Lieberman 2000, Neuron 28, 325-34; Carlsson et al, 2001, Annu. Rev. Pharmacol. Toxicol. 41, 237-60) and one of the major neurochemical theories is that dopaminergic dysfunction plays a crucial role in schizophrenia. An overactive dopaminergic system could result in symptoms of the disease. Therefore, many animal models used for schizophrenia research are created pharmacologically or genetically based on this dopamine hypothesis (Gainetdinov et al, 2001, Trends Neurosci. 24, 527-33). Amphetamine-induced hyperactivity and stereotypy is often used to model the positive symptoms of schizophrenia and the ability to reverse this behavior is considered a desired property of antipsychotic drugs (Segal D. S. et al, 1981, Essays Neurochem Neuropharmacol. 1981; 5:95-129. Review). Many neurotransmitters and receptors are known to be involved directly or indirectly via the dopamine (DA) system to affect the locomotor behavior of mice (Herz, 1998, Can. J. Physiol. Pharmacol. 76, 252-58). It is well known that dopaminergic activity can be probed in animal models by treatment with the psychostimulant, amphetamine, which exerts most of its effects in the central nervous system (CNS) through releasing biogenic amines and at least part of its locomotor stimulating action presumably is mediated by release of dopamine in the neostriatum. This lead us to the hypothesis that PKCI/HINT1 may play a role in schizophrenia by altering the dopaminergic function. To test this hypothesis, we studied the involvement of PKCI/HINT 1 in the hyperlocomotor activity of mice. We have looked at the involvement of PKCI at two levels of behavior: 1) The basal spontaneous locomotion, 2) Stimulated locomotor activity, modulated pharmacologically by D-amphetamine (AMPH). Initial experiments indicate that PKCI/HINT 1 KO mice displaced a relatively low level of spontaneous locomotion and an enhanced amphetamine-evoked locomotor response when compared with the wild type controls. Additional experiments addressed whether the effects of PKCI/HINT1 on amphetamine-evoked locomotor activity involved presynaptic or postsynaptic mechanisms. We used in vivo microdialysis to investigate whether PKCI/HINT1 deletion resulted in changes in basal and amphetamine-evoked extracellular DA. In addition we used apomorphine-evoked locomotor activity as a measure of postsynaptic dopaminergic activation. We also examined the distribution of PKCI/HINT1 in the brain regions that are known to have enriched dopaminergic neurons. Our results indicate that PKCI/HINT1 may play an important role in dopaminergic function and have important implications for the actions of psychostimulant as well as the neurobiology of schizophrenia. Moreover, the results suggest that PKCI/HINT1 regulates DA signaling at the postsynaptic level.

Finally, tests were used to assess depression and anxiety traits in PKCI/HINT1 wild-type and knockout mice as well as social cognition. Our data indicates that PKCI/HINT1 is present broadly throughout the regions of CNS with a relatively high abundance in olfactory system, cerebral cortex, hippocampus and part of thalamus, hypothalamus, midbrain, pons and medulla. Based on their distribution pattern, it is reasonable to speculate that in additional to dopaminergic system, PKCI also could be directly or indirectly involved with the function of other neurotransmission receptors or transporters, such as 5-HT, NE, Ach, GAGB.

Our results also revealed that PKCI KO mice showed a less depression and anxiety trait than their litter mate controls (WT), which indicate that PKCI could also play a role in regulating the emotion states of brain. Less depression/anxiety could represent as a part the symptoms of schizophrenia or they also could stand as the separate change of brain function due to the lack of PKCI gene in these mice. The psychobiological understanding of mood disorder is very limited and it seems involved with many different neurotransmission systems based on current pharmacological therapeutics. Our behavioral study was not able to eliminate the possibility that some neurotransmission systems other than dopamine are also contributing to the change. Therefore it further supports our speculation that PKCI could be directly or indirectly involved with the function of other neurotransmission receptors or transporters, such as 5-HT, NE, Ach, GAGB.

### SUMMARY OF THE INVENTION

The present invention relates to the involvement of PCKI/HINT1 in locomotor activity and its role in dopaminergic and central nervous system function.

Therefore, it is an object of the present invention to provide a method for increasing dopamine receptor(s) sensitivity, the method comprising reducing PKCI function or PKCI expression by providing a PKCI antagonist or inhibitor, or inhibiting or reducing the expression of PKCI at the RNA or protein level.

It is another object of the present invention to provide a composition for increasing dopamine receptor sensitivity, the composition comprising a PKCI function antagonist or inhibitor, or an inhibitor of PKCI RNA transciption or translation, or PKCI protein expression.

It is further an object of the present invention to provide a method for decreasing dopamine receptor sensitivity, the method comprising increasing PKCI function or PKCI expression by providing PKCI and/or increasing expression of PKCI at the RNA or protein level, or providing an agonist of PKCI or an enhancer or PKCI function.

It is yet another object of the present invention to provide a composition for decreasing dopamine receptor sensitivity, the composition comprising a PKCI agonist or enhancer, or an enhancer of PKCI RNA transcription or translation, or an enhancer of PKCI protein function.

It is another object of the present invention to provide a method for increasing dopamine receptor sensitivity through mediating a change in endogenous opioidergic function by reducing PKCI function or amount.

It is yet another object of the present invention to provide a method for decreasing dopamine receptor sensitivity through mediating a change in endogenous opioidergic function by increasing PKCI function or amount.

It is further an object of the present invention to provide methods for screening for agents which bind to, or modulate PKCI peptide, as well as the binding molecules and/or modulators, e.g. agonists and antagonists, particularly those that are obtained from the screening methods described herein.

It is another object of the present invention to provide a method for modulating glutamate receptors by modulating PKCI function or amount. It is expected that PKCI functions on the glutamatergic system.

It is yet another object of the present invention to provide methods for the treatment or prevention of neurobiological disorders, immune disorders, mood disorders, or cancers involving administering to an individual in need of treatment or prevention an effective amount of a purified antagonist or agonist of PKCI.

Glutamate receptors have been implicated in various neurological diseases and conditions, including, without limitation, schizophrenia, spinal cord injury, epilepsy, stroke, Alzheimer's disease, Parkinson's disease, Amotrophic Lateral Sclerosis (ALS), Huntington's disease, diabetic neuropathy, acute and chronic pain, ischemia and neuronal loss following hypoxia, hypoglycemia, ischemia, trauma, nervous insult, drug dependence and other compulsive disorders.

It is further an object of the present invention to provide a composition for reducing animal response to addictive drugs comprising an agonist of PKCI or additional PKCI RNA or peptide in an amount effective to reduce the action of addictive drugs in said animal said composition further comprising a pharmaceutically acceptable carrier, excipient, or diluent.

It is another object of the present invention to provide a method for modulating animal response to addictive drugs by modulating function or amount or both of PKCI. The KO mice seem to be more sensitive to the action of AMPH and cocaine which indicates that PKCI may suppress the action of addictive drugs. Therefore, by enhancing the function of PKCI or increasing the amount of PKCI, it may be possible to regulate the response to addictive drugs.

It is another object of the present invention to provide a model for studying schizophrenia, said model comprising PKCI knock-out mice which have been exposed to amphetamine or other psychostimulant which exerts its effects in the CNS by releasing dopamine.

It is a further object of the present invention to provide a treatment of schizophrenia, said treatment comprising a composition for modulating PKCI function or expression.

It is another object of the present invention to provide a treatment for mood disorders, e.g. depression or anxiety, said treatment comprising a composition for modulating PKCI function or expression.

It is another object of the present invention to provide a composition for treating schizophrenia comprising an effective amount of one or more modulators of PKCI function or expression in a pharmaceutically acceptable carrier, excipient, or diluent.

It is another object of the present invention to provide a composition for treating a mood disorder, e.g. depression or anxiety, comprising an effective amount of one or more modulators of PKCI function or expression in a pharmaceutically acceptable carrier, excipient, or diluent.

It is yet another object of the present invention to provide a method to identify mutations which confer susceptibility to illness by studying polymorphism on the PKCI gene, wherein a comparison between the gene in normal persons, i.e. persons without psychotic, mood and/or personality disorders, and the PKCI gene from persons with psychotic, mood and/or personality disorders, can identify mutations or polymorphisms in the gene that confer susceptibility to illnesses related to these disorders.

The disorders in these categories include, without limitation, schizophrenia, schizophreniform disorder, schizoaffective disorder, brief psychotic disorder, delusional disorder, shared psychotic disorder, psychotic disorder due to a general medical condition, substance-induced psychotic disorder, psychotic disorder not otherwise specified (American Psychiatric Association: Diagnostic and Statistical Manual of Mental Disorders, Fourth Edition, Washington, D.C., American Psychiatric Association, 1994).

The disorders in the category of Mood Disorders are: Major Depressive Disorder, Dyshtymic Disorder, Depressive Disorder Not Otherwise Specified, Bipolar I Disorder, Bipolar II Disorder, Cyclothymic Disorder, Bipolar Disorder Not Otherwise Specified, Mood Disorder Due to a General Medical Condition, Substance-Induced Mood Disorder, Mood-Disorder Not Otherwise Specified (American Psychiatric Association: Diagnostic and Statistical Manual of Mental Disorders, Fourth Edition, Washington, D.C., American Psychiatric Association, 1994).

The disorders in the category of Personality Disorders are: Paranoid Personality Disorder, Schizoid Personality Disorder, Schizotypal Personality Disorder, Antisocial Personality Disorder, Borderline Personality Disorder, Histrionic Personality Disorder, Narcissistic Personality Disorder, Avoidant Personality Disorder, Dependent Personality Disorder, Obsessive-Compulsive Personality Disorder, Personality Disorder NOS (American Psychiatric Association: Diagnostic and Statistical Manual of Mental Disorders, Fourth Edition, Washington, D.C., American Psychiatric Association, 1994).

The present invention also provides kits which are useful for carrying out the present invention. The present kits comprise a first container means containing any of the compositions mentioned above, for example, an agonist or antagonist of PKCI, or a compound which induces or inhibits PKCI expression or function. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

All the objects of the present invention are considered to have been met by the embodiments as set out below.

### BRIEF DESCRIPTION OF THE DRAWINGS

FIGS. 1A and 1B. spontaneous locomotion activity in the mPKCI^{-/-} and mPKCI^{-/-} mice during the light/dark phase of the cycle. Locomotion was monitored during the 30 minutes of acclimatization to the novel environment followed by a one-hour period. Counts per 5 minutes were averaged over the 30 minutes of acclimatization or the 1 h of spontaneous activity and mean+/−SEM are represented. A. Ambulation during acclimatization (for phase, F_{(1,26)}=119.21, p<0.0001; for genotype, F_{(1,26)}=30.07 p<0.0001) and during the following 1 hour (for phase, F_{(1,26)}=78.53, p<0.0001; for genotype, F_{1,26)}=39.84, p<0.000). B. Stereotypy during acclimatization (for phase, F_{(0,26)}=81.55, p<0.0001; for genotype, F_{(1,26)}=16.13 p=0.0004) and during the following one hour (for phase, F_{(0,26)}=44.85, p<0.0001; for genotype, F_{(0,26)}=12.77, p=0.0014). (*) p<0.05 and (***) p<0.0001 compare dark phase vs light phase; * p<0.05, ** p<0.01 and *** p<0.0001 compare mPKCI^{+/+} vs mPKCI^{-/-}; n=6-9 for mPKCI^{+/+}, n=6-9 for mPKCI^{-/-}; ANOVA followed by student's t test.

FIGS. 2A and 213. Acute morphine, bicuculline and D-amphetamine effect on ambulation and stereotypy. Saline (10 ml/kg i.p.), morphine (10 mg/kg i.p.), bicuculline (1 mg/kg i.p.) and D-AMPH (2.5 mg/kg i.p.) were administered after the period of acclimatization and locomotion was measured during the following 120 minutes. Results are expressed as mean of the counts over the 120 minutes+/−SEM. A. Ambulation (for treatment F_{(3,44)}=12.56, p<0.0001; for genotype F_{(1,44)}=3.37, p=0.0817). B. Stereotypy (for treatment F_{(3,44)}=11.46, p<0.0001; for genotype F_{(1,44)}=0.10, p=0.7542). (*) p<0.05 and (**) p<0.01 compare saline vs treatment; * p<0.05 and ** p<0.01 compare mPKCI^{+/+} vs mPKCI^{-/-}; n=5-6 for mPKCI^{+/+} and n=6-9 for mPKCI^{-/-}; ANOVA followed by student's t test.

FIGS. 3A and 3B. Dose response to acute D-amphetamine in mPKCI^{+/+} and mPKCI^{-/-} mice. Saline (10 ml/kg i.p.) or D-AMPH (1.25, 2.5 and 5 mg/kg i.p.) were administered after the period of acclimatization and locomotion was measured during the following 120 minutes. A. Ambulation (for treatment F_{(3,45)}=19.93, p<0.0001; for genotype F_{(1,45)}=7.83, p=0.0075). B. Stereotypy (for treatment F_{(3,45)}=18.52, p<0.0001; for genotype F_{(1,45)}=2.74, p=0.1048). (*) p<0.05, (**) p<0.01 and (***) p<0.0001 compares saline vs AMPH; (8) p<0.05 compares mPKCI^{+/+} vs mPKCI^{-/-}; n=6 for mPKCI^{+/+} and n=6-9 for mPKCI^{-/-}; ANOVA followed by student's t test.

FIG. 4. Basal and Amphetamine-evoked extracellular DA in the nucleus accumbens and caudate-putamen of mPKCI WT and KO mice. No net flux microdialysis was used to assess the status of extracellular DA concentration (DAext) nor in the extraction fraction (Ed) suggesting that both release and uptake of DA are unchanged in mPKCI KO mice. In addition the amphetamine-evoked DA response did not differ in WT and KO animals. The same results were obtained in both, the nucleus accumbens and the caudate-putamen.

FIGS. 5A and 5B. Ampomorphine-induced hyperlocomotion in mPKCI^{-/-} mice. Saline (10 ml/kg i.p.) or apomorphine (10 mg/kg i.p.) were administered after the period of acclimatization and locomotion was measured during the following 120 minutes. Results are expressed as mean+/−SEM of total scores. A. Ambulation (for treatment F_{(0,18)}=4.26, p=0.0538; for genotype F_{(1,18)}=1.55, p=0.2296). B. Stereotypy (for treatment F_{(1,18)}=49.32, p<0.0001; for genotype F_{(1,18)}=5.91, p=0.0257). (**) p<0.01 compares saline vs Apo; ** p<0.01 compares mPKCI^{+/+} vs mPKCI^{-/-}; n=5-6 for mPKCI^{+/+} and n=5-6 for mPKCI^{-/-}; ANOVA followed by student's t test.

FIGS. 6A, 6B, 6C. Forced swim test A. 4-month old mice, B. 6-month-old mice, C. 8.5-month-old mice. On day 1, KO (striped bar) animals of 4 (6A) and 8 months old show less immobility than their WT (solid bar) littermates. On day 2 that assess learning helplessness WT animals of all ages show an increase in immobility that reaches values of 200 seconds the last period. KO animals show a slight increase in immobility that anyway remains lower than in the WT. *p<0.05, **p<0.01, ***p<0.0001. Students' t-test WT vs KO.

FIG. 7. Tail suspension Test. WT, solid bar, PKCI/HINT1 knock-out mice, striped bar. F_{2,48}=1.37, p=0.2635 for age; F_{1,48}=111.22 p<0.0001 for genotype; F_{2,48}=19.31 p<0.0001 for interaction.

***p<0.001 Bonferroni WT vs KO; (*) p<0.05, (**) p<0.01, (***) Bonferroni vs 3 months.

FIG. 8. Effect of Haloperidol. WT, solid bar, PKCI/HINT1 knock-out mice, striped bar.

*p<0.05, **p<0.01; Bonferroni WT vs KO
(*)p<0.05, (**)p<0.01, (***)p<0.001; Bonferroni vs Saline. WT n=6, KO n=6.

FIG. 9. Dark-light test. WT, solid bar, PKCI/HINT1 knock-out mice, striped bar.

*P<0.05 Bonferroni WT vs KO; (*)p<0.05, (**)p<0.01 Bonferroni vs 5 min.

FIGS. 10A and 10B. A. Social approach test of adult males of 2-3 months old.***p<0.0001 Social vs non Social, Students't-test. WT n=5, KO n=5. B. Social approach of adult males of 9 months old. *p<0.05 Social vs non Social, Students't-test. WT n=6, KO n=7. Empty bar: non social; striped bar: social; stippled bar: neutral.

### DETAILED DESCRIPTION

The contents of all cited references (including literature references, issued patents, published patent applications, and co-pending patent applications) cited throughout this application are hereby expressly incorporated by reference.

### Definitions

Mutations or polymorphisms in PKCI polynucleotide include nucleic acid sequences containing deletions, insertions and/or substitutions of different nucleotides or nucleic acid sequences or genes resulting in a polynucleotide that is a functionally different polypeptide. Altered nucleic acid sequences can further include polymorphisms of the polynucleotide encoding the PKCI polypeptide; such polymorphisms are preferably detectable using a particular oligonucleotide probe. The encoded protein can also contain deletions, insertions, or substitutions of amino acid residues, which produce a silent change and result in a functionally nonequivalent PKCI protein.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNAs and can be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

The term “antibody” refers to intact molecules, as well as, fragments thereof, such as Fab, F(ab′)₂, Fv, or Fc, which are capable of binding an epitopic or antigenic determinant. Antibodies that bind to PKCI polypeptide can be prepared using intact polypeptides or fragments containing small peptides of interest, or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g, a mouse, a rat, or a rabbit).

An “agonist” refers to a molecule which, when bound to the PKCI polypeptide, or a functional fragment thereof, increases or prolongs the duration of the effect of the PKCI polypeptide, respectively. Agonists can include proteins, nucleic acids, carbohydrates, or any other molecules that bind to and modulate the effect of PKCI polypeptide. An antagonist refers to a molecule which, when bound to the PKCI polypeptide, or a functional fragment thereof, decreases or inhibits the amount or duration of the biological or immunological activity of PKCI polypeptide, respectively. “Antagonists” can include proteins, nucleic acids, carbohydrates, antibodies, or any other molecules that decrease or reduce the effect of PKCI polypeptide.

By modulators of the PKCI protein is meant agents which can affect the function or activity of PKCI in a cell in which PKCI function or activity is to be modulated or affected. In addition, modulators of PKCI can affect downstream systems and molecules that are regulated by, or which interact with, PKCI in the cell. Modulators of PKCI include compounds, materials, agents, drugs, and the like, that antagonize, inhibit, reduce, block, suppress, diminish, decrease, or eliminate PKCI function and/or activity. Such compounds, materials, agents, drugs and the like can be collectively termed “antagonists”. Alternatively, modulators of PKCI include compounds, materials, agents, drugs, and the like, that agonize, enhance, increase, augment, or amplify PKCI function in a cell. Such compounds, materials, agents, drugs and the like can be collectively termed “agonists”.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

Decreased or increased expression of the PKCI proteins of this invention can be measured at the RNA level using any of the methods well known in the art for the quantification of polynucleotides, such as, for example, PCR, RT-PCR, RNAse protection, Northern blotting and other hybridization methods. Assay techniques that can be used to determine levels of a protein, such as an PKCI protein, in a sample derived from a host are well known to those of skill in the art. Such assay methods include radioimmunoassays, competitive-binding assays, Western Blot analysis and ELISA assays.

To provide a basis for the diagnosis of disease associated with the expression of the PKCI protein, a normal or standard profile for expression is established. This can be accomplished by combining body fluids or cell extracts taken from normal subjects, either animal or human, with a sequence, or a fragment thereof, which encodes the PKCI polypeptide, under conditions suitable for hybridization or amplification. Standard hybridization can be quantified by comparing the values obtained from normal subjects with those from an experiment where a known amount of a substantially purified polynucleotide is used. Standard values obtained from normal samples can be compared with values obtained from samples from patients who are symptomatic for disease. Deviation between standard and subject (patient) values is used to establish the presence of disease.

Once disease is established and a treatment protocol is initiated, hybridization assays can be repeated on a regular basis to evaluate whether the level of expression in the patient begins to approximate that which is observed in a normal individual. The results obtained from successive assays can be used to show the efficacy of treatment over a period ranging from several days to months.

Methods suitable for quantifying the expression of PKCI include radiolabeling or biotinylating nucleotides, co-amplification of a control nucleic acid, and standard curves onto which the experimental results are interpolated (P. C. Melby, et al. J. Immunol. Methods, 159:235 244, 1993; and C. Duplaa, et al. Anal. Biochem., 229 236, 1993). The speed of quantifying multiple samples can be accelerated by running the assay in an ELISA format where the oligomer of interest is presented in various dilutions and a spectrophotometric or colorimetric response gives rapid quantification.

A variety of protocols for detecting and measuring the expression of the PKCI polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). These and other assays are described in the art as represented by the publication of R. Hampton et al., 1990; Serological Methods, a Laboratory Manual, APS Press, St Paul, Minn.; and D. E. Maddox et al., 1983; J. Exp. Med., 158:1211 1216).

Several assay protocols including ELISA, RIA, and FACS for measuring the PKCI polypeptide are known in the art and provide a basis for diagnosing altered or abnormal expression levels of the PKCI polypeptide. Normal or standard values for PKCI polypeptide expression are established by combining body fluids or cell extracts taken from normal mammalian subjects, preferably human, with antibody to the PKCI polypeptide under conditions suitable for complex formation. The amount of standard complex formation can be quantified by various methods; photometric means are preferred. Quantities of the PKCI polypeptide expressed in a subject sample, control sample, and disease sample from biopsied tissues are compared with the standard values. Deviation between standard and subject values establishes the parameters for diagnosing disease.

One embodiment of the present invention relates to the PKCI protein, antagonists, antibodies, agonists, complementary sequences, or vectors thereof of the present invention that can be administered in combination with other appropriate therapeutic agents for treating or preventing a neurological disease, mood disorder, or neurological disorder or condition. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one can achieve therapeutic efficacy with lower dosages of each agent, thus reducing the potential for adverse side effects.

In a further embodiment of the present invention, an antagonist or inhibitory agent of the PKCI polypeptide can be administered to an individual to prevent or treat a neurological disorder or mood disorder. Such disorders can include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder or mood disorder may be selected by testing for biological activity in promoting the survival or differentiation of neuron. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507 3515 (1990)); increased sprouting of neurons may be detected by methods known in the art, such as, for example, the methods set forth in Pestronk et al. (Exp. Neurol. 70:65 82 (1980)) or Brown et al. (Ann. Rev. Neurosci. 4:17 42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein).

Antagonists or inhibitors of the PKCI polypeptide of the present invention can be produced using methods which are generally known in the art. For example, an PKCI encoding polynucleotide sequence can be transfected into particular cell lines useful for the identification of agonists and antagonists of the PKCI polypeptide. Representative uses of these cell lines would be their inclusion in a method of identifying PKCI agonists and antagonists. Preferably, the cell lines are useful in a method for identifying a compound that modulates the biological activity of the PKCI polypeptide, comprising the steps of (a) combining a candidate modulator compound with a host cell expressing the PKCI polypeptide; and (b) measuring an effect of the candidate modulator compound on the activity of the expressed PKCI polypeptide. Representative vectors for expressing PKCI polypeptides are known in the art.

The cell lines are also useful in a method of screening for a compound that is capable of modulating the biological activity of the PKCI polypeptide, comprising the steps of: (a) determining the biological activity of the PKCI polypeptide in the absence of a modulator compound; (b) contacting a host cell expressing the PKCI polypeptide with the modulator compound; and (c) determining the biological activity of the PKCI polypeptide in the presence of the modulator compound; wherein a difference between the activity of the PKCI polypeptide in the presence of the modulator compound and in the absence of the modulator compound indicates a modulating effect of the compound. Additional uses for these cell lines are described herein or otherwise known in the art. In particular, purified PKCI protein, or fragments thereof, can be used to produce antibodies, or used to screen libraries of pharmaceutical agents to identify those which specifically bind PKCI.

Modifications of gene expression can be obtained by designing antisense molecules or complementary nucleic acid sequences (DNA, RNA, or PNA), to the control, 5′, or regulatory regions of the gene encoding the PKCI polypeptide, (e.g., signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence can also be designed to block translation of mRNA by preventing the transcript from binding to ribosomes.

Ribozymes, i.e., enzymatic RNA molecules, can also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding the PKCI polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site can be evaluated for secondary structural features which can render the oligonucleotide inoperable. The suitability of candidate targets can also be evaluated by testing accessibility to hybridization with complementary oligonucleotides using ribonuclease protection assays.

Complementary ribonucleic acid molecules and ribozymes according to the invention can be prepared by any method known in the art for the synthesis of nucleic acid molecules. Such methods include techniques for chemically synthesizing oligonucleotides, for example, solid phase phosphoramidite chemical synthesis. Alternatively, RNA molecules can be generated by in vitro and in vivo transcription of DNA sequences encoding the PKCI. Such DNA sequences can be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

In one embodiment of the present invention, an expression vector containing the polynucleotide encoding the PKCI polypeptide can be administered to an individual to treat or prevent a neurological disorder or mood disorder, including, but not limited to, the types of diseases, disorders, or conditions described above. Additionally, an expression vector containing the complement of the polynucleotide encoding the PKCI polypeptide can be administered to an individual.

Many methods for introducing vectors into cells or tissues are available and are equally suitable for use in vivo, in vitro, and ex vivo. For ex vivo therapy, vectors can be introduced into stem cells taken from the patient and clonally propagated for autologous transplant back into that same patient. Delivery by transfection and by liposome injections can be achieved using methods, which are well known in the art.

Any of the therapeutic methods described above can be applied to any individual or subject in need of such therapy, including but not limited to, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions can comprise the PKCI nucleic acid, antisense molecules, PKCI polypeptide or peptides, antibodies to the PKCI polypeptide, mimetics, agonists, antagonists, or inhibitors of the PKCI polypeptide or polynucleotide. The compositions can be administered alone, or in combination with at least one other agent, such as a stabilizing compound, which can be administered in any sterile, biocompatible pharmaceutical carrier, including, but not limited to, saline, buffered saline, dextrose, and water. The compositions can be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the PKCI nucleic acid, antisense, or polypeptide, or functional fragments thereof), the pharmaceutical compositions can contain suitable pharmaceutically acceptable carriers, diluents, or excipients comprising auxiliaries which facilitate processing of the active compounds into preparations which can be used pharmaceutically. Further details on techniques for formulation and administration are provided in the latest edition of Remington's Pharmaceutical Sciences (Mack Publishing Co.; Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents can be added, such as cross-linked polyvinyl pyrrolidone, agar, alginic acid, or a physiologically acceptable salt thereof, such as sodium alginate.

Dragee cores can be used in conjunction with physiologically suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances, which increase the viscosity of the suspension, such as sodium carboxymethyl cellulose, sorbitol, or dextran. In addition, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension can also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes.

The pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation can be a lyophilized powder which can contain any or all of the following: 1-50 mM histidine, 0.1% 2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of the PKCI product, such labeling would include amount, frequency, and method of administration.

Pharmaceutical compositions suitable for use in the present invention include compositions in which the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model can also be used to determine the appropriate concentration range and route of administration. Such information can then be used and extrapolated to determine useful doses and routes for administration in humans.

A therapeutically effective dose refers to that amount of active ingredient, for example, the PKCI polypeptide, or fragments thereof, antibodies to LRR polypeptides, agonists, antagonists or inhibitors of the PKCI polypeptide, which ameliorates, reduces, or eliminates the symptoms or condition. Therapeutic efficacy and toxicity can be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀ Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The practitioner, who will consider the factors related to the individual requiring treatment, will determine the exact dosage. Dosage and administration are adjusted to provide sufficient levels of the active moiety or to maintain the desired effect. Factors, which can be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions can be administered every 3 to 4 days, every week, or once every two weeks, depending on half-life and clearance rate of the particular formulation. Variations in these dosage levels can be adjusted using standard empirical routines for optimization, as is well understood in the art.

Normal dosage amounts can vary from 0.1 to 100,000 micrograms (ug), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

Another embodiment of the invention embraces a method of screening for compounds capable of modulating the activity of PKCI. One technique for drug screening provides for high throughput screening of compounds having suitable binding affinity to the protein of interest as described in WO 84/03564 (Venton, et al.). In this method, as applied to the PKCI protein, large numbers of different small test compounds are synthesized on a solid substrate, such as plastic pins or some other surface. The test compounds are reacted with the PKCI polypeptide, or fragments thereof, and washed. The bound PKCI polypeptide is then detected by methods well known in the art. Purified PKCI polypeptide can also be coated directly onto plates for use in the aforementioned drug screening techniques. Alternatively, non-neutralizing antibodies can be used to capture the peptide and immobilize it on a solid support.

In a further embodiment of this invention, competitive drug screening assays can be used in which neutralizing antibodies, capable of binding the PKCI polypeptide, specifically compete with a test compound for binding to the PKCI polypeptide. In this manner, the antibodies can be used to detect the presence of any peptide, which shares one or more antigenic determinants with the PKCI polypeptide, respectively.

Other screening and small molecule (e.g., drug) detection assays which involve the detection or identification of small molecules or compounds that can bind to a given protein, i.e., the PKCI polypeptide, are encompassed by the present invention. Particularly preferred are assays suitable for high throughput screening methodologies. In such binding-based screening or detection assays, a functional assay is not typically required. All that is needed is a target protein, preferably substantially purified, and a library or panel of compounds (e.g., ligands, drugs, small molecules) to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP; Exton, Pa.) as described in U.S. Pat. Nos. 6,020,141 and 6,036,920 to Pantoliano et al.; see also, J. Zimmerman, 2000, Gen. Eng. News, 20(8)). The assay allows for the detection of small molecules (e.g., drugs, ligands) that bind to expressed, and preferably purified, PKCI polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

Other features of the invention will become apparent in the course of the following descriptions of exemplary embodiments which are given for illustration of the invention and are not intended to be limiting thereof.

The following MATERIALS AND METHODS were used in the examples that follow.

PKCI^{-/-} mice

The generation of PKCI^{-/-} mice was described previously (Su et al, 2003, supra). PKCI^{-/-} mice and their wild type littermates were derived by breeding heterozygous PKCI^{-/-} mice for altered PKCI allele (Hint1) and genotype of the animals was confirmed by PCR of DNA from tail biopsies. Animals were housed 4-6/cage, maintained under standard laboratory condition with food and water provided ad libitum. The male animals were tested between 10-20 weeks of age. Wild type and PKCI^{-/-} groups were matched for age in all experiments. All studies were conducted with an approved protocol from University of Maryland, School of Pharmacy IACUC.

Locomotor Activity Measurement

Mice locomotor activity was monitored during an open field test using “Activity Monitor” chambers (27×27×20.3 cm) associated with the Activity Monitor software (Med Associates Inc St Albans Vt.). Room temperature was set at 23° C.+/−2° C. Horizontal spontaneous locomotion activity, scored as ambulatory counts and stereotypic movements (Sanberg P. R. et al 1987, Pharmacol. Biochem. Behav. 27, 569-72), was recorded during the first 30 min of acclimatization to the novel environment and during 120 min following the treatment. The animals were acclimated to a 12 h cycle light/dark phase with the light on at 7:00 am before the experiments. Tests were performed during the light phase of the cycle between 8:00 am to 3:00 pm. AMPH or other drugs was prepared freshly before each experiment by dissolving in saline (NaCl 0.9%) and administered via intra peritoneal in a total volume of 10 ml/kg at the indicated doses.

In Vivo Microdialysis

PKCI^{-/-} mice and their WT controls were anesthetized and implanted unilaterally with a microdialysis guide cannula (CMA/11, CMA microdialysis) in the nucleus accumbens (AP: +1.5, L: −0.8, V: −3.8 from bregma) or the dorsal striatum (AP: +0.4, L−2.1, V−2.2 from bregma) using standard stereotaxic techniques, and allowed to recover for 5 days prior to the microdialysis experiment as described. After recovery, the microdialysis probe (CMA/11, CMA microdialysis, North Chelmsford, Mass.) was connected to the dialysis system, flushed with artificial cerebrospinal fluid (aCSF: 145 mM NaCl, 2.8 mM KCl, 1.2 mM CaCl₂, 1.2 mM MgCl₂, 0.25 mM ascorbic acid, and 5.4 mM D-glucose, pH 7.2 adjusted with NaOH 0.5 M), and slowly inserted into the guide cannula. The dialysis system consisted of FEP tubing (CMA microdialysis) that connected the probe to a 1 ml gastight syringe (Hamilton Co., Reno, Nev.) mounted on a microdialysis pump (CMA/102) through a quartz-lined, low resistance swivel (375/D/22QM, Instech, Plymouth Meeting, Pa.). After probe insertion, the mouse was placed in the dialysis chamber with food and water freely available, and the probe perfused overnight with aCSF at a flow rate of 0.6 μl/min. The next morning, the perfusion syringes were loaded with fresh aCSF and probes were allowed to equilibrate for an additional 1 hour prior to the commencement of experiments. A flow rate of 0.6 μl/min was used for all the studies.

For no net flux experiments, five different concentrations of DA (0, 5, 10, 20 and 40 nM) in aCSF were perfused in random order through the dialysis probe. Each DA concentration was perfused for 30 min, and then 2×10 min samples were collected. Following completion of the no net flux experiments, normal aCSF was again perfused through the probe for 30 min. allowing for a period of equilibration. Consecutive 15 minutes samples were then collected. After three baseline samples mice received a saline ip injection and three more samples were collected. Then mice received a 2.5 mg/kg ip d-amohetamine injection and samples were collected for an additional 90 minutes. Samples were stored at −80° C. until analysis. The DA content was determined by HPLC coupled to electrochemical detection with an external standard. All the samples were analyzed within 48 hours of collection. After the experiment, mice were sacrificed by a pentobarbital overdose and their brains were removed, frozen on dry ice and 20 μm sections were obtained on a cryostat for the histological verification of probe location. No net flux data was analyzed as described (Chefer et al 2006, supra). The net flux of DA through the probe (DAin-DAout) was calculated and plotted against the concentration of DA perfused (DAin). The following parameters were calculated from the resulting linear function. The Y-axis intercept, corresponding to zero DA perfused through the probe is the dialysate DA concentration (DAdial) in a conventional microdialysis experiment. The X-axis intercept corresponds to the point of equilibrium where there is no net flux of DA through the probe and reflects an estimation of the extracellular DA concentration (DAext). Finally the slope of the regression line corresponds to the extraction fraction (Ed) which is a measure of the ability of the tissue to extract DA and has been shown to be an indirect measure of DA uptake (Smith and Justice 1994, J. Neurosci. Methods 54, 75-82; Chefer et al 2006, supra). In the conventional microdialysis experiments investigating the effects of amphetamine, the average of the three baseline samples was calculated, and all the DA concentrations were expressed as % of baseline. Differences between WI and KO animals in the appropriate variables was asses by comparing both groups using a Student's t test.

Immunohistochemistry

Animals and preparation of tissue: mPKCI wild-type (mPKCI^{+/+}) mice and mPKCI gene knockout (mPKCI^{-/-}) mice were used in the present study. The adult mice were anesthetized with 7.5 mg ketamine hydrochloride (Pfizer AB, Sweden) and 2.5 mg xylazine (Veterinaria AG, Switzerland) per 100 g body weight intra-peritoneally. Animals were perfused transcardially with saline and then with 4% paraformaldehyde (PFA) in phosphate buffer (0.1 M, pH 7.4) for 10 min. The whole brain was removed, post-fixed in 4% PFA at 4° C. overnight, equilibrated with 30% sucrose in phosphate buffer at 4° C. for 48h. The whole brain was embedded with O.C.T. (Tissue-Tek, Sakura Finetek U.S.A. Inc.) and cut coronally into 25 μm sections using a cryostat (OTF5000, Bright, Jencons, UK). All sections were kept with long term protecting solution at 4° C. until used.

Floating sections were incubated with 1% hydrogen peroxide in 70% methanol-tris buffered saline (TBST; 0.1 M Tris, pH 7.4, 0.9% saline, and 0.3% Triton X-100) for 30 min at room temperature (all incubations were performed at 22-25° C.) to inhibit endogenous peroxidase followed by three times wash with TBST and 1 h incubation in 1% bovine serum albumin (BSA)-TBST. Then, the sections were incubated with hPKCI antiserum diluted 1:10000 in TBST containing 1% BSA for 24 h, washed in TBST (three washes of 5 min duration each), incubated for 1 h in 1:1000 dilution of biotinylated donkey anti rabbit serum (Biotin-SP-Conjugated Donkey Anti-Rabbit Ig G (H+L), Jackson ImmunoResearch Lab, Inc. West Grove, Pa., USA), rinsed, and finally visualized with ABC reagent (ABC Kit PK-6100, Vector, USA) according to manufacturer's manual and mounted onto gelatin coated slides, coverslipped with DPX mounting medium.

Slides were viewed and imaged using Nikon E800 microscopes and Nikon digital camera. Images were edited using Photoshop (v CS; Adobe Systems Inc., San Jose, Calif., USA).

For immunofluorescence staining, sections were incubated with 1% bovine serum albumin (BSA)-1% normal donkey serum (NDS) in tris buffered saline (TBST; 0.1 M Tris, pH 7.4, 0.9% saline, and 0.3% Triton X-100) for 1 h at room temperature (all incubations were performed at 22° C.), then incubated in hPKCI antiserum diluted 1:1000 in TBST containing 1% BSA for 24 h, washed in TBST (three washes of 5 min duration each), followed by incubation with 1:500 dilution of Cy2 conjugated donkey anti-rabbit antibody (Jackson ImmunoResearch Lab, Inc. West Grove, Pa., USA) for 1 h. Then they were rinsed and incubated with a mixture of 1:1000 diluted mouse anti-NeuN (Neuronal Nuclei, Chemicon,) and 1:500 diluted Goat anti-GFAP (Glial fibrillary acidic protein, Santa Cruz Biotechnology, Inc.) in 1% BSA/TBST for 24 h and then washed in TBST as above. The sections were incubated with a mixture of Cy5 conjugated donkey anti-mouse antibody and Cy3 conjugated donkey anti-goat antibody (Jackson) 1:500 in 1% BSA/TBST for 60 min, washed in TBST as above, incubated with 2 nM DAPI (4′-6-Diamidino-2-phenylindole, Molecular Probes) for 60 min, washed in TBS, and coverslipped with fluorescence mounting medium.

Slides were viewed under Nikon E800 microscope and images were captured on a Fluo View X confocal microscope (Olympus Instruments, CA, USA).

Statistical Analysis

Analyses of variance (ANOVA) were used to compare the results of the behavioral experiments. Post-hoc comparisons between groups were made using student t-test with Welch's correction when applies. Data are presented as mean+/−SEM. Statistical analysis were performed using Graphpad Prism version 4.00 for Windows (Graphpad Software, San Diego Calif., USA)

### Example 1

### PKCI KO Mice with Lower Spontaneous Locomotor Activity Displayed Supersensitive Response to Amphetamine

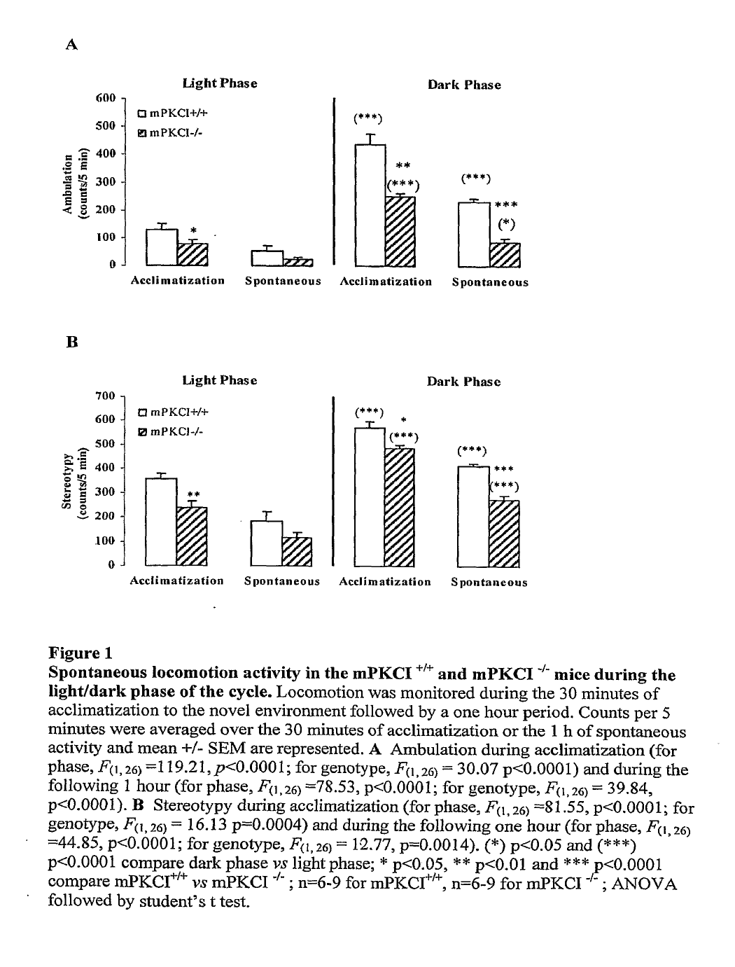

The spontaneous locomotor activity measured during the light phase of the cycle (between 8:00 am to 3:00 pm) and during the dark phase of the cycle (between 9:00 pm to 4:00 am), which corresponds to the active phase, is shown in FIG. 1. During the light phase, WT and KO mice display a lower level of spontaneous activity, measured as distance traveled and stereotypic movement for 120 min. In the dark phase, both genotypes exhibit an increase of locomotion as expected. However, the KO mice consistently scored on average 40% lower than the WT in either the light or the dark phase. These results indicate that the KO mice are hypolocomotive both under habituated basal conditions (light/dark phases) or during exploration of a novel environment (acclimatization phase) in comparison with the WT mice.

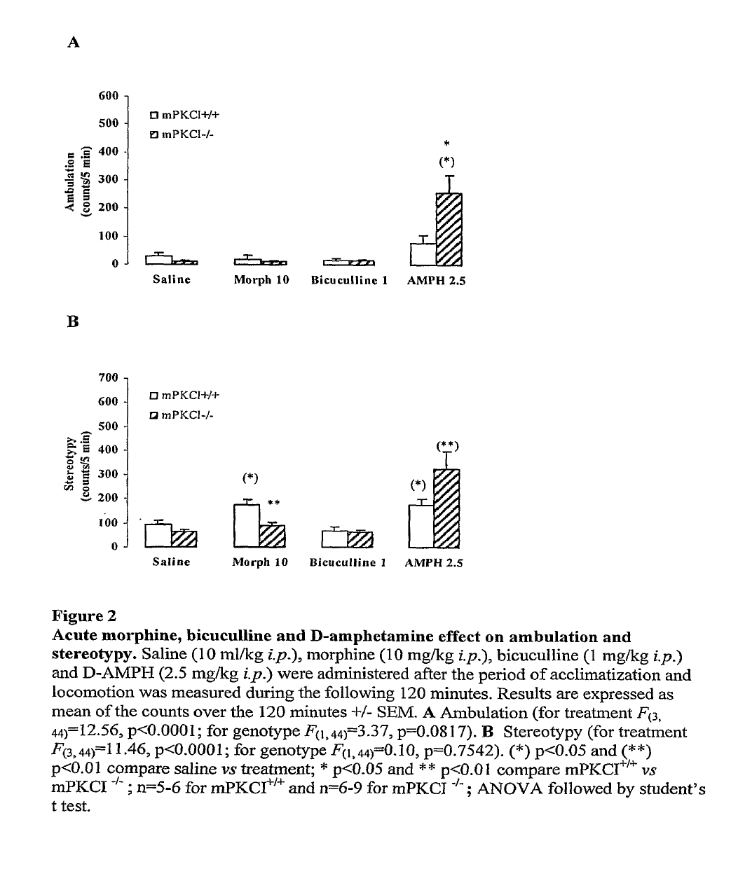

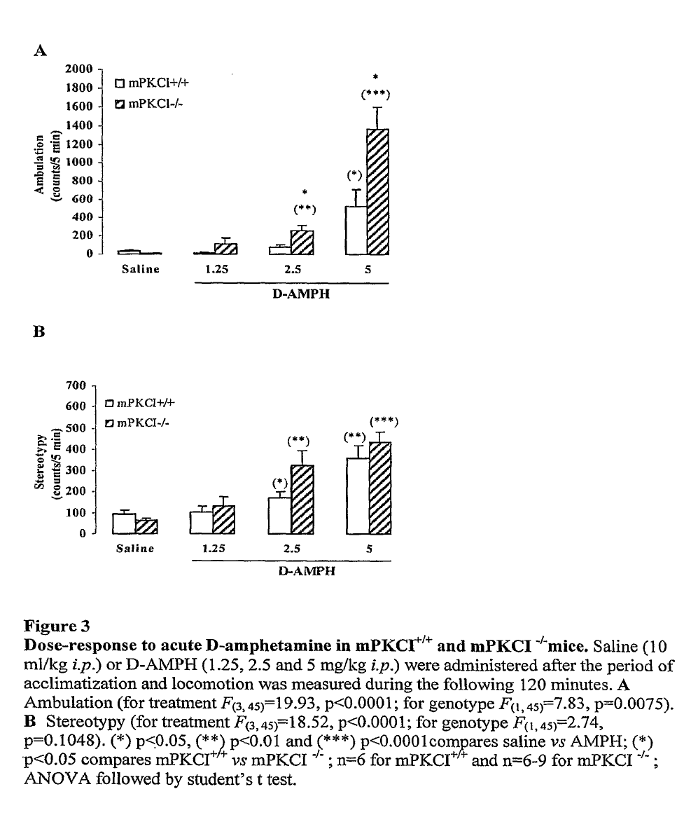

Rodent locomotor activity is known to be affected by many drugs with CNS stimulant actions. The effect of amphetamine on the locomotor activity of mPKCI KO mice was examined in this study and morphine, bicuculline are also included as non-dopaminergic controls. As shown in FIG. 2, morphine and bicuculline at a dose of 10 mg/kg and 1 mg/kg respectively did not promote increase in locomotor activity but AMPH at a dose of 2.5 mg/kg increased locomotion in both WT and KO animals. However, PKCI KO mice displayed an enhanced three times more amphetamine-evoked locomotor response as compared with the WT animals. The increase in amphetamine-evoked activity in mPKCI KO mice was consistent across a range of amphetamine doses as shown in FIG. 3. Although most of the amphetamine's locomotor stimulating action including stereotyped behavior is presumably mediated by amphetamine-induced DA release from dopaminergic nerve terminals, amphetamine is also known to alter other biogenic amines. We next tested the effects of a specific dopamine transporter blocker, GBR 12909, in the locomotion test. Interestingly, GBR 12909 produced a comparable enhancement of locomotion in mPKCI KO mice as amphetamine (data not shown). These results indicated that mice without mPKCI/HINT1 are supersensitive to amphetamine and this enhanced response is likely mediated through the dopaminergic system since it is mimicked by selective DA transporter blockers.

### Example 2

### Extracellular Dopamine Levels in Nucleus Accumbens and Dorsal Striatum

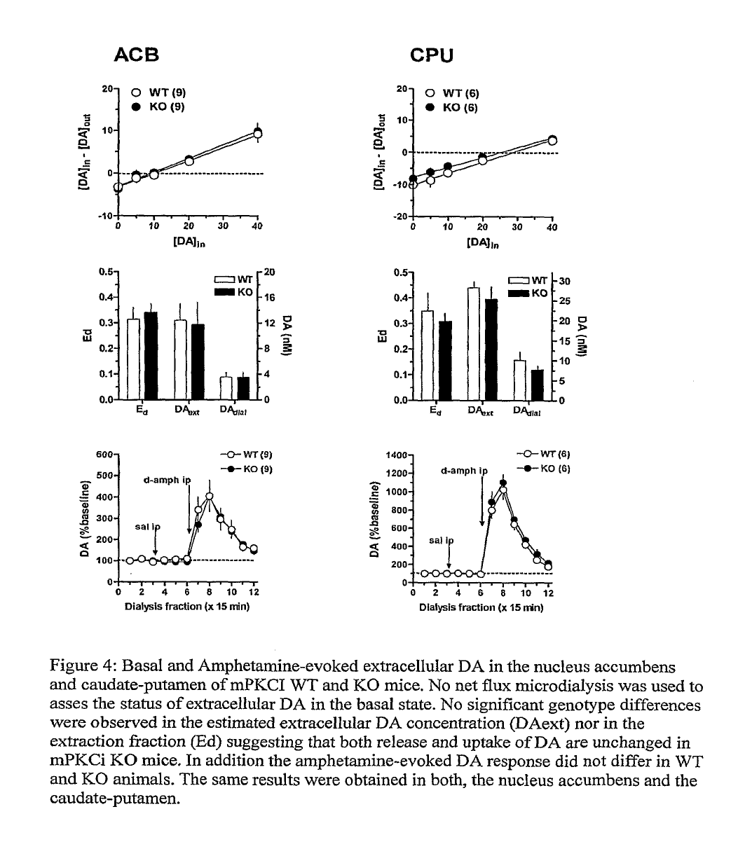

DA projections to the striatum play an important role in the control of locomotor activity. Therefore, we used the technique of in vivo microdialysis in order to investigate the consequences of genetic deletion of the mPKCI protein on basal extracellular DA dynamics as well as on the amphetamine-evoked DA response. Quantitative no net flux microdialysis indicated that there were no significant differences in basal dialysate (DA dial) levels nor in the estimated extracellular DA concentration (DAext) (FIG. 4). Moreover, the DA extraction fraction (Ed), calculated as the slope of the no net flux regression line was unchanged in KO mice, suggesting that deletion of the mPKCI/HINT1 did not alter the clearance of extracellular DA by the DA transporter (Smith and Justice 1994, supra; Chefer et al 2006, supra). Similar results were obtained in both the nucleus accumbens and the caudate-putamen. In addition, we investigated the ability of amphetamine to increase dialysate DA using conventional microdialysis. No significant difference between WT and KO mice was found in the amphetamine-evoked DA response neither in the dorsal (caudate-putamen) nor ventral striatum (nucleus accumbens). The dose of amphetamine used in the microdialysis studies was previously found to induce an enhanced locomotor response in the KO mice. The lack of genotype differences in the amphetamine-evoked DA response suggests that the enhanced behavioral sensitivity to amphetamine in the KO mice is due to changes at the postsynaptic rather than presynaptic level.

### Example 3

### Apomorphine Induced a Differential Hyperlocomotor Activity in mPKCI KO Mice

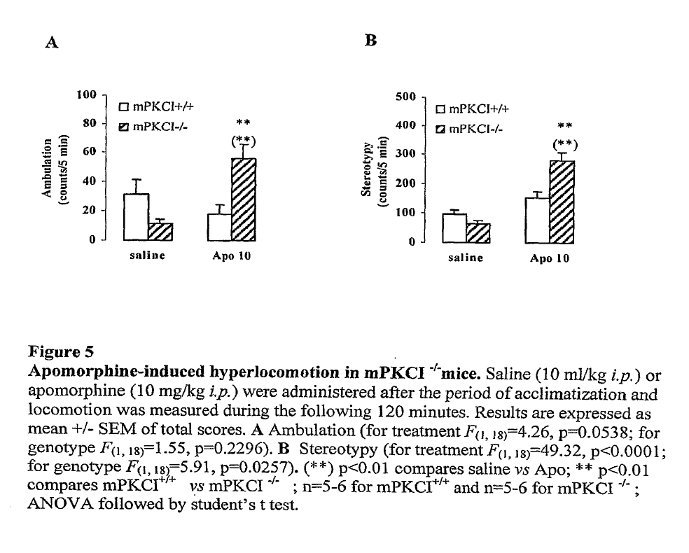

To further explore the mechanism responsible for the behavioral supersensitivity to amphetamine observed in mPKCI KO mice, we tested the locomotor response to the non-selective dopamine receptor agonist, apomorphine. A high dose (10 mg/kg) was used in order to probe postsynaptic DA receptor function. Data shown in FIG. 5 revealed that mPKCI KO mice responded with significantly higher locomotor activity as compared with WT in both total ambulation and stereotyped behavior. This result indicates that mPKCI/HINT1 deletion results in postsynaptic DA receptor supersensitivity. This observation suggests that the enhanced behavioral sensitivity to amphetamine observed in mPKCI mice is most likely through a modified postsynaptic mechanism.

### Example 4

### mPKCI/HINT1 Brain Distribution and Neuronal Expression

To determine the expression of PKCI/HINT 1 in central nervous system samples of mouse brain cortex, cerebellum, midbrain and spinal cord, taken from PKCI KO and WT mice were examined by Western blotting (data not shown). The Western blot revealed a broad expression pattern of mPKCI/HINT 1 in mouse brain and spinal cord, suggesting an important role for mPKCI/HINT 1 in neurological system. The distribution of PKCI/HINT 1 immunoreactivity in the brain tissue sections was identified in patterns consistent with neurons and neuronal processes in various brain regions including frontal cortex and striatum (data not shown). There was only low level background staining in brain sections from PKCI/HINT1 gene knockout mice (data not shown). Detailed distributions of PKCI/HINT1 within neurons and neuronal processes was viewed more dearly in the triple-labeled fluorescent immunostaining in paraformaldehyde-fixed frozen brain sections (data not shown). The PKCI/HINT 1 immunoreactivity was observed in neurons labeled with specific anti-neuronal marker antibody (anti-NeuN), but was absent from astrocytes stained with specific anti glia marker antibody (anti-GFAP). A low level of non-specific background reactivity was noted as a light green color in sections from both WT and KO mice, however, this did not interfere the observation of specific immunoreactivity. These anatomic results may help to place PKCI/HINT1 in the appropriate cellular context for the interpretation of the results observed in the behavioral study.

### Example 5

### Two Different Tests were Used to Assess Depression Traits in PKCI/HINT1 Wild Type and Knockout Mice, the Forced Swim Test or Porsolt's Test and the Tail Suspension Test

### A-Forced Swim Test

The forced swim test is a test of learned behavioral despair (Porsolt RD et al., 1977, Nature 269, 730-732). Mice are placed in an opaque 5 L cylinder (40 cm high, 25 cm diameter) filled with 3.5 L of 30° C. water where they swim without the opportunity to escape or touch the bottom. The time spent immobile is recorded. Immobility is monitored when the mouse is only making movements necessary to keep the head above the water and maintains a stationary posture for 2 seconds. In this posture the mouse's forelimbs are motionless and directed forward, the tail is directed outward and the hind legs are in limited motion. Animals showing difficulty in swimming or in staying afloat are excluded.

The test is a two days procedure. On day 1 the mice are placed in water to swim for a single trial of 15 min immobility is recorded in the last 4 min of the trial. On day 2 the mice are placed in water through a series of four trials of 6 min each; immobility is recorded in the last 4 min. Each trial is followed by an 8 min rest when the animals are dried with towels and returned to their cages. Table 1 shows the number of animals used in each age group.

TABLE 1

| | 4 | 6 | 8.5 |
|---|---|---|---|
| n | months | months | months |
| WT | 12 | 5 | 6 |
| KO | 12 | 5 | 7 |

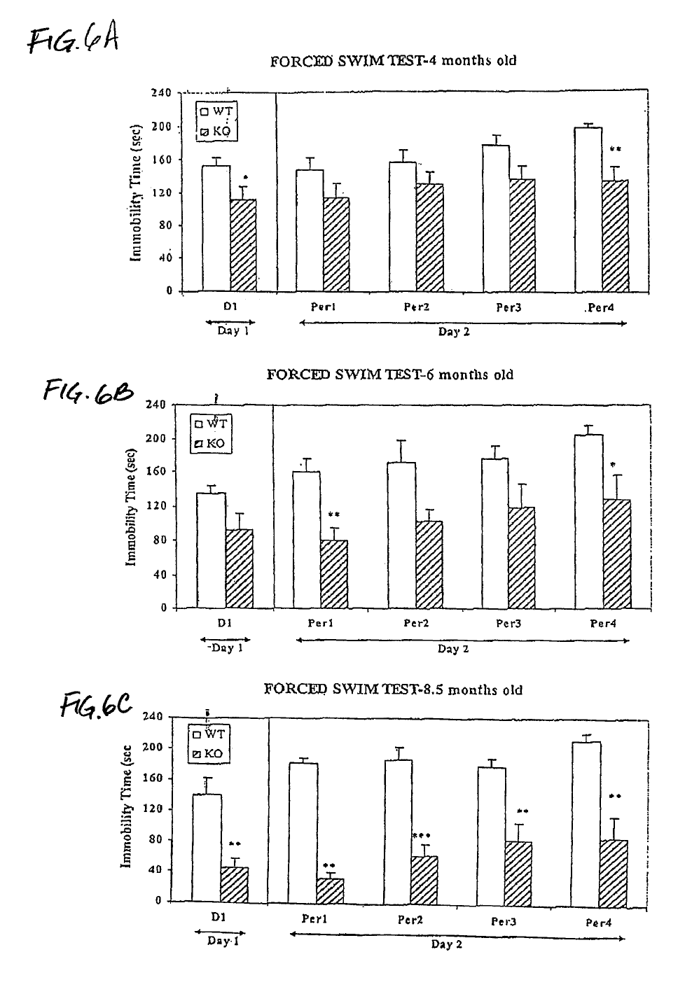

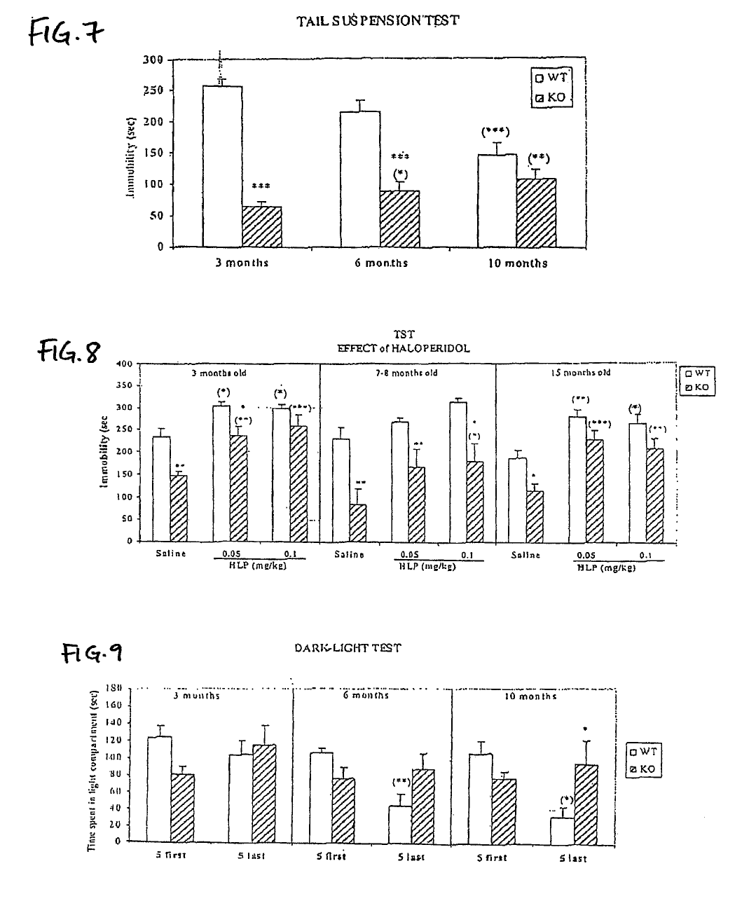

Our results indicate that on day 1, KO animals of 4 months (FIG. 6A) and 8 months (FIG. 6C) of age show less immobility that their WT littermates. On day 2 that assesses learning helplessness, WT animals of all ages show an increase in immobility that reaches values of 200 seconds the last period. KO animals show a slight increase in immobility that anyway remains lower that in WT.

### Tail Suspension Test

In order to confirm the results of the forced swim test while removing any bias introduced by the animal's swimming or learning capacity, we used the tail suspension test. This test assesses depression trait using coping behavior in a stressful situation (Cryan J F et al., 2005, Neruosc. Behav. Rev. 29, 571-625). The animals are exposed to a haemodynamic stress of being hung in an uncontrollable fashion by the tail for six minutes. Immobility, considered as a depression trait when the animal gives up any escape, is reported in seconds. Data are expressed as mean+/−SEM and analyzed using a 2 way ANOVA (age×genotype) followed by Bonferroni when p<0.05.

Results indicate that PKCI/HINT1 KO mice show less depression trait than their wild type littermates (FIG. 7). Wild type animal behavior shows that immobility time is dependent on the age; 10 months old animals display 40% less immobility than 3 months old one. When compared to their wild type littermates, knock out animals display 4 times less immobility at 3 months old and 2 times less immobility at 6 months old (p<0.001-Bonferroni). Table 2 shows the number of animals used in the experiment in each age group.

TABLE 2

| | 3 | 6 | 10 |
|---|---|---|---|
| n | months | months | months |
| WT | 10 | 10 | 6 |
| KO | 10 | 11 | 7 |

Therefore, results of forced swim test and tail suspension test show that KO animal display less depression traits than their WT littermates.

### Effect of Haloperidol

In order to probe whether the dopaminergic system is involved in the decrease in immobility displayed by the PKCI/HINT1 KO mice, Haloperidol (Sigma) was used. This antipsychotic that inhibits the D2, D3, D4 dopaminergic receptors was previously described to increase mice immobility in the tail suspension test (Steru et al., 1987, Prog. Neuropsychopharmacol. Biol. Psychiatry 11, 659-71). Haloperidol 0.05 mg/kg and 0.1 mg/kg were administered via intra-peritoneal (10 ml/kg) 30 minutes before the test. Data are expressed as mean+/−SEM and analyzed using a 2 way ANOVA (treatment×genotype) followed by Bonferroni when p<0.05.

Haloperidol 0.05 mg/kg and 0.1 mg/kg increased immobility in both WT and KO animals when compared with saline treatment (FIG. 8). At both 3 months and 15 months old, a dose of 0.1 mg/kg haloperidol induced an increase in immobility in KO mice, reaching the level attained by WT animals.

The difference between WT and KO mice in the tail suspension test response can be abolished using the dopaminergic antagonist haloperidol.

### Example 6

### Test of Anxiety Trait: Dark-Light Avoidance Test

This test of light avoidance and dark preference assesses anxiety trait (Kromer S A et al., 2005, J. Neuroscience 25, 4373-4384; Klemenhagen K C et al., 2006, Neuropsychopharmacology 31, 101-111). The dark-light box consisted in two compartments (13.95 cm×27.9 cm) one black colored the other transparent, separated with an open door, the ensemble forms an insert. The insert was placed into an open field chamber equipped with 16 infra-red beams allowing tracking the animal position; the test was automated using activity monitor software (Med Associates, St Albans Vt.). Mice were placed in the box for 30 minutes; the total amount of time spent by the animal in each compartment was monitored by 6 periods of 5 minutes each and reported in seconds. Results are expressed as mean+/−SEM of time spent in the light compartment during the five first minutes and the five last minutes of the test (FIG. 9).

For all ages WT animals show a decrease in the time spent in the light compartment, over the time, which is significant at 6 and 10 months old. KO animals of same age do not show any decrease in time spent in the light compartment. Thus KO animals seem to show less anxiety trait over the time in comparison with their WT littermates. Table 3 shows the number of animals used in each age group.

TABLE 3

| | 3 | 6 | 10 |
|---|---|---|---|
| n | months | months | months |
| WT | 9 | 8 | 6 |
| KO | 13 | 8 | 7 |

### Social Approach

This test assesses social cognition impairment in mice in relation with social withdrawal that is one of the negative symptoms of Schizophrenia (Green et al., 2005, Schizophrenic Bulletin 31, 882-887).

The method used is derived from Moy et al 2004 (Genes, Brain & Behavior 3, 287-302), using automated open field apparatus as described by Nadler et al 2004 (Genes, Brain & Behavior 3, 303-314). Adult male test mice (2 months and more) are isolated for 48 h prior to the test.

The testing apparatus was a rat open field chamber divided in a 3-chambered compartments with doorways in dividing walls. The system was automated using Activity monitor software (Med Associates—St Albans Vt.). The coordinates of the compartments are described in table 4.

TABLE 4

| | | Number of |
|---|---|---|
| Rat Open Field | Coordinates | beams |
| Compartment 1 | 0.5 to 7 | 7 |
| Compartment 2 | 7 to 9 | 2 |
| Compartment 3 | 9 to 16 | 7 |

Compartments 1 and 3 are social or non social, compartment 2 with a smaller size is called neutral.

In a pre-test of 10 minutes male mouse is placed the neutral compartment and allowed to explore all three compartments. Time spent in each compartment is monitored. The compartment in which the animal spends more time is assigned as non social compartment.

Prior to the test of a juvenile (28 days old) stimulus mice is placed in the social compartment. During the test the adult mouse is placed in the neutral compartment and allowed to explore all three compartments for 20 minutes. Time spent in each compartment is monitored. Results are expressed as percent time spent in each compartment and are presented as mean+/−SEM.

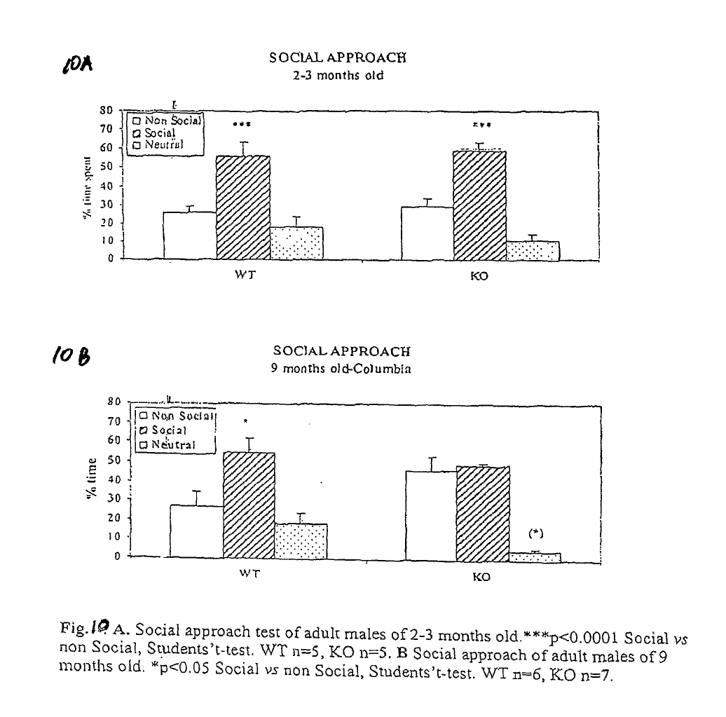

WT animals (n=5) of 2 months (FIG. 10A) and 9 months (n=6) (FIG. 10B) show preference for the social compartment. Young KO animals of 2 months (n=5) show preference for the social compartment. Older KO mice of 9 months old (n=7) spent the same amount of time in the social and the non social compartments, thus do not show any social preference.

### Discussion

Locomotion is one of the most common behaviors in which rodent engage, and thus the assessment of locomotor activity is an essential component of animal behavioral analyses. Neurological input is required for initiation and ongoing control of locomotion. Psychostimulant amphetamine produces profound motor activity and this property has been used to model the psychotic symptoms of the schizophrenia. Many proteins in CNS are known or speculated to play important roles in the control of locomotion and response to psychostimulant actions, for example, biogenic amine transporters and receptors, glutamate receptors; GABA receptors, opioid receptors or other proteins that may regulate the function of these neurotransmission systems (Gainetdinov et al, 2001, supra). This study has potentially identified a novel player, mPKCI, adding to this list based on the observations from the study of mPKCI KO mice. Since the function of mPKCI protein is not certain as described in the introduction, its effect on the locomotion is a surprise but exciting news, and the supersensitivity to amphetamine in mPKCI KO mice provides an interesting phenotype for further study of mPKCI function in CNS. Finding the potential functional phenotype of PKCI/HINT1 is also of significant clinical relevance since this protein has been one of the candidate protein molecules that has been identified from schizophrenia patients (Vawter et al, 2001, Brain Res Bull. 2001 Jul. 15; 55(5):641-50).

Psychostimulants like AMPH elicit locomotor activity, rewarding and reinforcement via the stimulation of DA release in the NAc (Zahniser N. R. & Sorkin A. 2004, Neuropharmacology 47, Suppl. 1, 80-91). The existence of a tonically active MOR system that stimulates mesoaccumbems DA neurotransmission is a well known phenomenon (Spanagel et al, 1992, Proc. Natl. Acad. Sci. USA 89, 2046-50; Herz, 1998, Can. J. Physiol. Pharmacol. 76, 252-58). Within this context, several possible explanations for the present observations may be drawn: 1, PKCI might have a direct modulating role within the DA system (interact with DA receptors or DA transporters), or 2. The supersensitivity to AMPH could be mediated through the change of endogenous opioidergic function caused by lack of PKCI, which is plausible since PKCI/HINT1 seems to be involved with the mu opoioid receptor (Guang et al, 2004, Mol. Pharmacol. 66, 1285-92). If the second explanation is true, one may ask why morphine, a MOR preferred ligand, did not produce any increased locomotion in the PKCI KO mice. Indeed, morphine seems to have a depressive locomotor effect on both wild type and knockout mice in this study. From literature reports, both stimulative and depressive locomotor effects of morphine have been observed, depending on the dose, the interval after the administration (Patti et al, 2005, Pharmacol. Biochem. Behav. 81, 923-27), and strain of the mice (Belknap et al, 1998, Pharmacol. Biochem. Behav. 59, 353-60). At the 10 mg/kg dose (also used in our study), a decrease in locomotion was often observed (Patti et al, 2005, supra). Thus, it is plausible that the PKCI induced opioidergic functional changes that underlie the locomotor activity alteration, may be more readily observed under AMPH because of its strong locomotor stimulation action.

The primary targets of AMPH in the CNS are the monoamine transporters. AMPH induces release of DA through the DA transporter (Sulzer et al, 1993, J. Neruochem. 60, 527-35; Schenk, 2002, Prog. Drug. Res. 59, 111-31) and this effect is critical for AMPH induced behavioral activation since mice lacking the DA transporter are insensitive to the locomotor stimulant effects of amphetamines (Giros et al, 1996, Nature 379, 606-12). Thus, the enhanced behavioral sensitivity to AMPH in mPKCI KO mice may be explained by an enhanced AMPH evoked DA overflow in NAc and PCU. However the in vivo microdialysis data from this study did not support this possibility, suggesting that increased presynaptic DA release is unlikely the cause for the behavioral super sensitivity to AMPH observed in mPKCI KO mice. Other explanations for this supersensitive behavior need to be explored. The apomorphine result did provide a clue that pointed to the postsynaptic sites of the dopamine neurons. However, the mechanism for the postsynaptic modification in the mPKCI KO mice remains to be determined. Change of the expression of dopamine receptors, alterations in receptor signal transduction, modification of the receptor, and phosphorylation all can lead to a functional consequence that is observed from this study. The result from the previous study (Guang et al 2004, Mol. Pharmacol. 66, 1285-92) showed that mPKCI inhibited PKC related MOR phosphorylation. Although it is not clear whether the inhibition of MOR phosphorylation in mPKCI expressing cells is due to the direct inhibition of PKC activity or some indirect way, that result suggests that PKCI could play an important role in regulation of neurotransmitter receptors phosphorylation. A previous study (Namkung and Sibley, 2004, J. Biol. Chem. 279, 49533-41) suggested that PKC mediated phosphorylation of D₁ and D₂ receptors would lead to increasing of the receptor sequestration, while lacking or attenuated receptor phosphorylation would lead to less internalization. Thus, it is plausible to speculate that mPKCI/HINT1 might have an inhibitory function on the D₂ receptors phosphorylation and release of such an inhibition, for example, by deleting the mPKCI gene, would cause an increase of the receptor internalization. Although internalization has been thought to contribute directly to functional desensitization of receptor signaling by rapidly reducing the number of receptors present at the cell surface, it has been proposed that internalization also mediates receptor resensitization (Law et al, 2000, Mol. Pharmacol. 58, 388-98; Koch et al, 1998, J. Biol. Chem. 273, 13652-57). Therefore the consequence of receptor phosphorylation could lead to an overall enhanced receptor function.

Furthermore, the result of our immunohistochemistry study of mPKCI/HINT1 revealed that the expression of the protein is primarily localized to neurons, which is consistent with the result of in situ hybridization analysis of HINT1 mRNA (Vawter et al, 2004, supra), and provided a good neuroanatomic basis of the potential function of mPKCI/HINT1 in CNS. Previous study on the intracellular localization of PKCI/HINT1 protein revealed that PKCI/HINT1 was present mainly in the nucleus with lesser amount in the cytoplasm. However, the intracellular localization study (Klein, et al, 1998, Exp. Cell. Res. 244, 26-32) was carried out on the non-neuronal cells. Our results of the neuronal cellular distribution have shown that mPKCI/HINT1 in neurons is primarily in the cytoplasma and neural process, indicating the expression patterns of mPKCI/HINT 1 in neuronal cells and non-neuronal cells are different, which also indicates that the function of mPKCI/HINT1 in neurons may be distinct from its peripheral counterpart.

The mPKCI/HINT1 KO mice displayed a relative hypolocomotion status compared to the WT mice during at normal or novel environment in this study. However, the D-AMPH-induced locomotor activity in KO mice is substantially higher than WT animals. These data suggest that the motor function of the KO animal is likely normal but their responses to AMPH are significantly enhanced. The hypolocomotive phenotype could not be readily explained by the hypothesis that DA receptors function is enhanced in mPKC/HINT1 I KO mice and is subject to further study.

Microarray analysis of gene expression is a very effective approach to examine the global changes in the various physiological or disease state. Via this approach PKCI/HINT1 was identified as one of down regulated genes from samples of schizophrenic patients (Vawter et al, 2004, supra). However, finding the functional implication would be crucial in determining if these changes are actually involved in the pathophysiology of schizophrenia. The finding from the current study is consistent in several aspects, the alteration of gene expression and the localization of gene expression, with the microarray study therefore has provided a strong functional evidence to support the notion that PKCI/HINT1 may play a role in schizophrenia. This could lead to several interesting questions regarding future research, such as how the dopaminergic function is affected by PKCI/HINT1, whether the PKCI KO mice can be appropriate as a genetic model for schizophrenic study, and whether glutamate neurotransmission function, which is another major neurotransmitter that is implicated in the disease state of schizophrenia, is or can be altered in PKCI KO mice. Considering that there are some limitations for using conventional KO mice in order to study the specific function of a gene, such as the presence of gene alteration in all the tissues that naturally express the gene, these limitations could cause great difficulty when assigning a behavioral variation to a specific brain structure or pathway and the compensatory changes in other genes could occur in animals after gene alteration. Using the additional different approaches to verify and further examine the PKCI/HINT1 function in CNS would be required for the better understanding of its role in schizophrenia.

Our data indicates that PKCI/HINT1 is present broadly throughout the regions of CNS with a relatively high abundance in olfactory system, cerebral cortex, hippocampus and part of thalamus, hypothalamus, midbrain, pons and medulla. Based on their distribution pattern, it is reasonable to speculate that in additional to dopaminergic system, PKCI also could be directly or indirectly involved with the function of other neurotransmission receptors or transporters, such as 5-HT, NE, Ach, GAGB.

In addition, Our results also revealed that PKCI KO mice showed a less depression and anxiety trait than their litter mate controls (WT), which indicate that PKCI could also play a role in regulating the emotion states of brain. Less depression/anxiety could represent as a part the symptoms of schizophrenia or they also could stand as the separate change of brain function due to the lack of PKCI gene in these mice. The psychobiological understanding of mood disorder is very limited and it seems involved with many different neurotransmission systems based on current pharmacological therapeutics. Our behavioral study was not able to eliminate the possibility that some neurotransmission systems other than dopamine are also contributing to the change. Therefore it further supports our speculation that PKCI could be directly or indirectly involved with the function of other neurotransmission receptors or transporters, such as 5-HT, NE; Ach, GAGB.

Finally, we are looking at the involvement of PKCI in the rewarding function in PKCI KO mice. In this study, we are testing the response of the KO mice to the reward action of amphetamine and cocaine by using conditional place preference test. Our preliminary data indicates that the KO mice show a higher response to amphetamine in this behavioral test. The implication of this result is that the PKCI may be able to regulate the animal response to addictive drugs.

Other research involves the PCP effect on PKCI KO mice on locomotion, social behavior of the KO mice. This study is designed to examine if PKCI has any functional role in glutamatergic system. If this protein is indeed involved with glutamatergic system as its expression pattern in CNS suggested, we should observe the differential changes of locomotion and social behavior after animal received PCP, which has actions primarily on glutamate receptors. The positive results from this study would indicate that PKCI could also regulate the function of glutamatergic system.

1. A method for increasing dopamine receptor sensitivity, said method comprising reducing PKCI function wherein PKCI function is reduced by any of the following: reducing or inhibiting PKCI RNA expression, reducing or inhibiting PKCI protein expression, and providing a PKCI inhibitor or antagonist.

2. (canceled)

3. A composition comprising a PKCI inhibitor or antagonist.

4-5. (canceled)

6. A method for decreasing dopamine receptor sensitivity, said method comprising increasing PKCI function wherein PKCI function is increased by any of the following: increasing or enhancing PKCI RNA expression, increasing PKCI protein expression or stability, providing a PKCI enhancer or agonist.

7. (canceled)

8. A composition comprising a PKCI enhancer or agonist.

9-10. (canceled)

11. The method of claim 1 wherein said reduction in PKCI function mediates a change in endogenous opioidergic function.

12-13. (canceled)

14. A composition for use in the method of claim 11 wherein said composition comprises any of: an inhibitor of PKCI RNA, an inhibitor of PKCI protein expression or function, and a PKCI antagonist.

15. The method of claim 6 wherein said increase in PKCI function mediates a change in endogenous opioidergic function.

16. A composition for use in the method of claim 15 wherein said composition comprises an enhancer or agonist of PKCI.

17. (canceled)

18. A composition for use in the method of claim 15 wherein said composition comprises an enhancer of PKCI RNA and/or protein expression.

19. A model for studying schizophrenia said model comprising PKCI knock-out mice which have been exposed to a psychostimulant which exerts its effects by releasing dopamine.

20. The model of claim 19 wherein said stimulant is amphetamine or a derivative thereof.

21. A method for identifying mutations which confer susceptibility to psychotic, mood and/or personality disorders, said method comprising studying polymorphisms on the PKCI gene, wherein a comparison between the gene in normal persons, i.e. without psychotic, mood and/or personality disorders, and the PKCI gene from persons with said disorders, can identify polymorphisms or mutations in the gene that confer susceptibility to illness related to said disorders.

22. The method of claim 21 wherein said personality disorder is schizophrenia.

23. The method of claim 21 wherein said mood disorder is chosen from the group consisting of depression and anxiety.

24. A kit for identifying one or more polymorphisms associated with a disease condition said kit comprising means necessary to identify polymorphisms in PKCI gene.

25. The kit of claim 24 wherein said disease condition is chosen from the group consisting of schizophrenia, depression, and anxiety.

26. (canceled)

27. A composition for the treatment of a condition associated with PKCI expression or function, said composition comprising one or more modulators of PKCI expression or function.

28. The composition of claim 27 wherein said condition is chosen from the group consisting of schizophrenia, depression, and anxiety.

29. (canceled)

30. A method for treating or reducing symptoms of a condition associated with PKCI expression or function, said method comprising modulating PKCI expression or function such that symptoms are reduced.

31. The method of claim 30 wherein said condition is chosen from the group consisting of schizophrenia, depression and anxiety.

32. (canceled)
